(19) United States
(12) Patent Application Publication
(10) Pub. No.: US 2024/0354868 A1
(43) Pub. Date: Oct. 24, 2024

(54) SOFTWARE AND METHOD FOR MENTAL WELLNESS

(71) Applicant: Micheal Wayne Olson, Peace River (CA)

(72) Inventor: Micheal Wayne Olson, Peace River (CA)

(21) Appl. No.: 18/304,252

(22) Filed: Apr. 20, 2023

(51) Int. Cl. G06Q 10/105; G16H 50/20

(52) U.S. Cl. CPC G06Q 50/01; G06Q 10/105; G16H 50/20

(57) ABSTRACT

A software application that is configured to facilitate a method operable to assist in the mental health of an individual whom has been subjected to workplace violence or harassment. The present invention includes a method that is configured to allow a user to anonymously join a user group of other individuals who have also endured similar experiences. The software application of the present invention provides assignment of a category of the experience in order to group individuals based on similar experiences. The user provides input of their experience in order to receive feedback from either a group of other users or a single user. The software application further provides assessment of a user state of the user wherein the user state is correlated to the user's ability to cope with the situation with which they have dealt with at their workplace.

### FIELD OF THE INVENTION

The present invention relates generally to a mental wellness software application and method, more specifically but not by way of limitation, a software application that facilitates a method of connecting individual's whom have been subjected to violence or harassment in the workplace wherein the method of the present invention includes but is not limited to sharing of experiences with others.

### BACKGROUND

Workplace bullying is verbal, physical, social or psychological abuse by your or manager, co-worker or a group of people at work. Workplace bullying can happen in any type of workplace, from offices to shops, cafes, restaurants, workshops and community groups Workplace bullying can even happen to volunteers, students, interns, apprentices, in addition to permanent employees. Some types of workplace bullying are criminal acts and violate local laws that can be reported directly to the police. Workplace bullying is performed in a variety of manners and some examples are: repeated hurtful remarks; making fun of an individual or their work; sexual harassment, which can include unwelcome touching and sexually explicit comments; excluding an individual or stopping an individual from working with people or taking part in activities that relates to your work; playing mind games with a worker or other types of psychological harassment; intimidation which often makes a worker feel less important and undervalued; requiring an individual to perform impossible jobs that can't be completed in the given time or with the resources provided and deliberately changing an individual's work hours or schedule to make it difficult for the worker. These are just a few examples of workplace harassment and bullying. These behaviors can have a negative impact on an individual's ability to work as well their personal health. Some negative impacts are: be less active or successful; lower confidence in work; feelings of being scared, stressed, anxious or depressed; negative impact on life outside of work; a lack of trust in your employer or co-workers; a lack confidence and happiness about work or contribution to the organization and can further manifest into physical signs of stress like headaches, backaches, sleep problems.

Harassment at the workplace is a serious issue and affects millions of people every year. In order for harassment to occur one must understand what type of behavior is considered harassment and when is a law potentially violated. First, unlawful harassing conduct must be un-welcome and based on the victim's protected status. Second, the conduct must be: subjectively abusive to the person affected; and objectively severe and pervasive enough to create a work environment that a reasonable person would find hostile or abusive. Prohibited workplace harassment may take either of two forms. It may entail quid pro quo harassment, which occurs in cases in which employment decisions or treatment are based on submission to or rejection of unwelcome conduct, which can include but is not limited to conduct of a sexual nature. Workplace harassment may also consist of offensive conduct based on one or more of the protected groups identified by most local laws that is severe or pervasive wherein it creates a hostile or offensive work environment or when it results in an adverse employment decision.

Whether an instance or a pattern of harassing conduct is severe or pervasive is determined on a case-by-case basis, with consideration paid to the following factors: the frequency of the unwelcome discriminatory conduct; the severity of the conduct; whether the conduct was physically threatening or humiliating, or a mere offensive utterance; whether the conduct unreasonably interfered with work performance; the effect on the employee's psychological well-being; and whether the harasser was a superior within the organization. Each factor is considered, but none are required or dispositive. Hostile work environment cases are often difficult to recognize, because the particular facts of each situation determine whether offensive conduct has crossed the line from ordinary tribulations of the workplace, such as the sporadic use of abusive language and occasional teasing, to unlawful harassment. The impacts to individuals creates serious mental and physical health issues and many times these go untreated for various reasons.

It is intended within the scope of the present invention to provide a software application that facilitates a method of connecting individual's whom have been subjected to violence or harassment in the workplace.

### SUMMARY OF THE INVENTION

It is the object of the present invention to provide a software that is configured to facilitate connection of individuals whom have been subjected to workplace violence or harassment wherein the software application of the present invention is operable on a smart phone or other portable computing device.

Another object of the present invention is to provide a method for connecting individuals having a shared experience of workplace harassment wherein the present invention provides categorization of different types of workplace harassment.

A further object of the present invention is to provide a software that is configured to facilitate connection of individuals whom have been subjected to workplace violence or harassment wherein the method of the present invention groups individuals together based on category of workplace harassment.

Still another object of the present invention is to provide a method for connecting individuals having a shared experience of workplace harassment wherein the method of the present invention includes an ability for a user to input their experience.

An additional object of the present invention is to provide a software that is configured to facilitate connection of individuals whom have been subjected to workplace violence or harassment wherein the present invention further provides information on the legal rights for an individual exposed to workplace violence or harassment.

Yet a further object of the present invention is to provide a method for connecting individuals having a shared experience of workplace harassment wherein the software application further provides connection to mental health practitioners for individuals.

Another object of the present invention is to provide a software that is configured to facilitate connection of individuals whom have been subjected to workplace violence or harassment wherein the software application assigns a mental health status to a user and tracks progress through stages thereof.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is an outline of the method of the present invention.

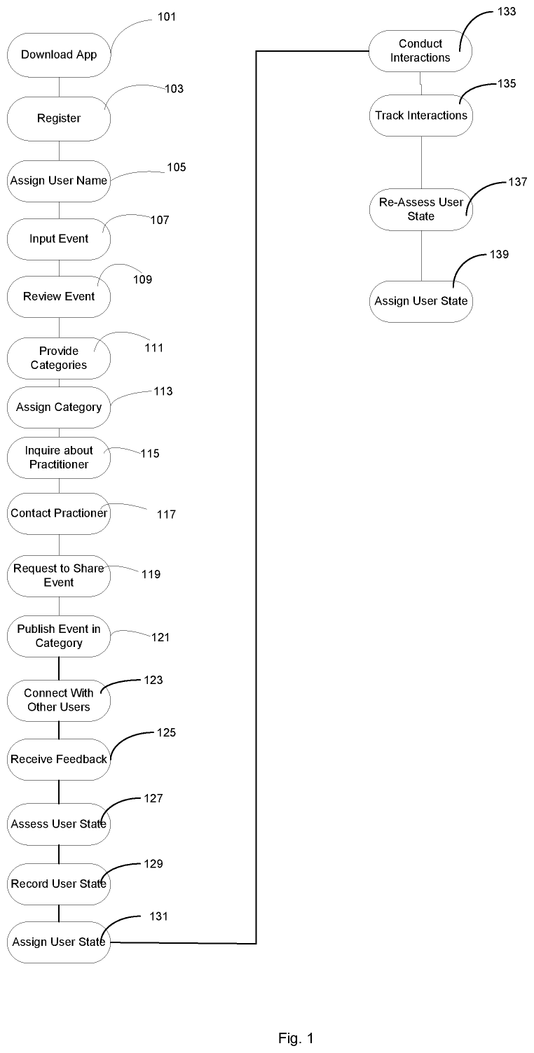

### DETAILED DESCRIPTION

Referring now to the drawings submitted herewith, wherein various elements depicted therein are not necessarily drawn to scale and wherein through the views and FIGURES like elements are referenced with identical reference numerals, there is illustrated a method for mental wellness 100 constructed according to the principles of the present invention.

An embodiment of the present invention is discussed herein with reference to the FIGURES submitted herewith. Those skilled in the art will understand that the detailed description herein with respect to these FIGURES is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular to the FIGURE submitted herewith, an outline of the method for mental wellness 100 is diagrammed therein. In step 101, a user will download the software application of the present invention to a suitable computing device such as but not limited to a smart phone. Step 103, the user will register within the software application to create a profile wherein the registration process captures demographic data and other pertinent data about the user. In step 105, the software application will assign the user an anonymous name/user identification in order to protect the privacy of the user. It is also contemplated within the scope of the present invention that the user can create their own anonymous name/user identification. Step 107, the user will input the details of the event that occurred at their workplace. It is contemplated within the scope of the present invention that the input of the details of the event of the user is facilitated by a question set having a combination of multiple choice answers as well as input of text from the user.

In step 109, the software application will scan the inputted data from step 107. Step 111, the software application will provide to the user the suggested categories for which the categorize the inputted event. It should be understood within the scope of the present invention that the categories could be divided into categories such as but not limited to defamation of character, workplace bullying and sexual harassment. In step 113, the user will agree to the suggested category or select a desired category that is appropriately defines the type of event the user experienced. Step 115, the software application will prompt the user if the user desires to be connected to a legal or mental health practitioner. In step 117, if the user has selected to be connected to a legal or mental health practitioner the software application will transmit the contact information of the user to the appropriate legal and/or mental health practitioner. Step 119, the software application prompts the user with a request to share the inputted event from step 107. Step 121, if the user agrees to share the event, the event of the user is published wherein the user identification of the user is associated therewith. The event is published in the category that was assigned thereto.

In step 123, the user can then receive feedback and advice from other users that have been associated with the same category. It should be understood within the scope of the present invention that the feedback could be provided in either a group format or in a one to one communication wherein all feedback is provided through the software application. In step 125, the user receives the feedback from either a group or a single individual. Step 127, the software application will assess the state of the user. It should be understood within the scope of the present invention that the assessment of the user state relates to their mental stability and ability to cope with the event in which they have inputted into the software application. This assessment is executed with the software application and questions presented to the user. In step 129, the user will have their user state recorded and stored into a database. Step 131, the assigned user state is associated with the profile of the user. In step 133, the software application will facilitate the conduction of subsequent interactions between the user and either groups or other individuals that have inputted data into the same category as the user. Step 135, the software application of the present invention tracks and records the interactions of the user with the groups and/or individuals in which they have engaged therewith. In step 137, the software application will provide a re-assessment of the user state through presentation of an additional questionnaire. The user will answer the questions provided thereto and the data will be analyzed by the software application of the present invention. Step 139, based on the analyzed answers to the questions answered in step 137, the user will be assigned a second user state wherein if applicable will be a different user state than the original user state assigned. It should be understood within the scope of the present invention that the software application provides a group of user states wherein the user states range from a low user state, meaning the individual is having a difficult time coping, to a high user state wherein the individual is demonstrating an ability to deal with the workplace event that they have experienced.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A method of addressing mental wellness for individuals that have been exposed to workplace violence and harassment wherein the method of the present invention comprises:
downloading a software application, said software application being downloaded by a plurality of users onto a remote computing device;
registering with the software application, wherein the plurality of users create user profiles within the software application;
assigning a user name, wherein the plurality of users are given anonymous user identification to be employed while utilizing the software application;
inputting data regarding a workplace event, wherein the users will input data that provides information about a workplace event that can be qualified as violent or harassment;
reviewing the data, wherein the software application executes a review of the data;
determining a category for the data, wherein the software application provides a determination of the data so as to suggest a category therefore;
providing a suggested category to the user, wherein the software application provides a category to the user that defines the event that occurred at their workplace;
assigning a category to the event, wherein the user is assign a category for their event on which the user input data thereabout;
inquiring for a practitioner connection, wherein the software application prompts the user for connection to a professional practitioner;
assessing a user state, wherein the software application through a questionnaire assesses a user's ability to cope with the event;
requesting to share the event, wherein the software application requests the user to share the data on the event;
publishing the data on the event, wherein the data on the event is published and listed by category to all other users;
receiving feedback from at least one user, wherein the user associated with the data on the event receives feedback from at least one user concerning the event;
assessing the user state, wherein the software application assesses the user state ensuing the user receiving feedback;
tracking the user state, wherein the software application provides tracking of the user state for the user;
reassessing the user state, wherein ensuing a plurality of feedback the software application reassesses the user state of the user; and
changing the user state of the user, wherein the software application changes the user state to a next level of progression of improvement.

2. The method of addressing mental wellness for individuals that have been exposed to workplace violence and harassment as recited in claim 1, wherein the step of inquiring for a practitioner connection further includes potential connection to a legal practitioner or a mental health practitioner.

3. The method of addressing mental wellness for individuals that have been exposed to workplace violence and harassment as recited in claim 2, and further including a step of publishing an event by category, wherein the software application publishes the event in a specific category.

4. The method of addressing mental wellness for individuals that have been exposed to workplace violence and harassment as recited in claim 3, and further including the step of recording an initial user state of the user, wherein the initial state is an indication of a user's ability to cope with the event.

5. The method of addressing mental wellness for individuals that have been exposed to workplace violence and harassment as recited in claim 4, and further including a step of allowing a user to create a user profile identification.

6. The method of addressing mental wellness for individuals that have been exposed to workplace violence and harassment as recited in claim 5, and further including the step of conducting subsequent interactions between the plurality of users to discuss the event.
